(19) United States
(12) Patent Application Publication
(10) Pub. No.: US 2011/0117596 A1
(43) Pub. Date: May 19, 2011

(54) COMPOSITE SORBENT MATERIAL, ITS PREPARATION AND ITS USE

(76) Inventors: Bo Mattiasson, Hjarup (SE); Fatima Plieva, Lund (SE)

(21) Appl. No.: 12/293,633

(22) PCT Filed: Mar. 20, 2007
(86) PCT No.: PCT/SE2007/050175

§ 371 Date: Oct. 29, 2008

(30) Foreign Application Priority Data
Mar. 21, 2006 (SE) 0600632-4

(51) Int. Cl. C12P 1/00; B01D 15/08; B01D 53/02; B01J 20/28; B01J 20/26; B01J 20/02; B01J 20/24; C12N 11/08; C12N 11/10; C12N 11/12; C12N 5/071

(52) U.S. Cl. 435/41; 210/635; 95/88; 502/400; 502/402; 502/405; 502/403; 502/404; 502/401; 435/180; 435/178; 435/179; 435/397

(57) ABSTRACT

A composite sorbent material is disclosed which comprises A) a housing of a dense, insoluble material having openings therein and B) a porous polymer gel housed within said housing, said housing being provided with openings therein for fluid communication between said porous monolithic polymer gel and the surroundings, and said porous monolithic polymer gel occupying the inner space of said housing and being kept on place within said housing by mechanical means. Processes for the preparation of composite sorbent materials by cryoprecipitation are also disclosed as well as the use of the composite sorbent material according to the invention e.g. for the removal of pollutants from gases or liquids, for the enrichment or separation of molecules and for cell culture.

### TECHNICAL FIELD

The present invention relates to a composite sorbent material, processes for its preparation and the use of such a composite sorbent material. More particularly, the present invention relates to a composite sorbent material based on a porous polymer gel as the sorbent active principle, processes for the preparation of such a composite absorbent material and the use of said composite absorbent material e.g. for the removal of pollutants from gases or liquids, for the enrichment or separation of molecules and for cell culture.

### BACKGROUND ART

Sorption processes (using suitable cheap sorbents) are by far the most widely used technique in environmental area for the removal of different pollutants from water. Therefore it is essential to develop low cost technologies evolving use of sorbents.

A number of conventional treatment technologies have been considered for treatment of wastewater contaminated with organic substances. Among them, adsorption is found to be the most effective method. Activated carbon (AC) is widely used for adsorptions of organics because of its capability for efficiently adsorbing a broad range of different types of adsorbates. However its use is limited, because of its high cost.

Removal of heavy metals in waste water is achieved by ion exchange, chemical oxidation, chemical precipitation and biological removal. A lot of work has been done in the area of synthesis and applications of polymeric chelating ion exchangers. However the high cost of the developed chelating sorbents has been a major obstacle for their wide use in environmental applications. Ion-exchangers and inorganic adsorbents are widely applied to water and waste water treatment plants. Economical anion-exchangers were produced from waste natural materials (WNM). WNM (examples are coconut husk, sugarcane bagasse, tealeaf, rice hull etc) were converted successfully into anion-exchangers through chemical reactions with thionylchloride, dimethylamine, DMF as solvent and formaldehyde as crosslinking agent (Orlando et al., 2003).

Solid phase extraction (SPE) is one of the most used technologies, capable of isolating and enriching the trace metals from aqueous samples (Fontanals et al., 2005; Rossi and Zhang, 2000). The most important sorbents used for SPE are silica with chemically bonded various groups (Poole, 2003), carbons (Masque et al., 1998), and macroporous styrene-divinylbenzene (St-DVB) sorbents (Fontanals et al., 2005).

One promising concept for multi-pollutant control is the circulating fluidized bed absorber (CFBA), which may utilize injection of different sorbents to remove multiple pollutants (Mao et al., 2004). For example, a CFBA could scrub SO₂ through adsorption onto calcinated lime and could eliminate mercury vapor through adsorption onto activated carbon. Increased solids-gas contact and solids recycling promises both efficient pollutant removal and economy. A potential side benefit is the capture of fine particulate matter through agglomeration onto sorbent particles (Mao et al., 2004).

All the sorbents mentioned above are mainly in the traditional spherical beads shape and are supplied by many companies (for the sorbents for SPE see reviews (Fontanals et al., 2005; Poole, 2003)). However some sorbents caused leaking and back-pressure problems; another sorbent showed a lot-to-lot variability in particle size (Fontanals et al., 2005).

A further disadvantage of prior art sorbent materials is their sensitivity to shear-forces caused by stirring when used in a CFBA.

Accordingly there is a continued need for a sorbent material which can be prepared at a reasonable cost and be used in a CFBA without being rapidly degraded due to shear-forces caused by stirring.

### SUMMARY OF THE INVENTION

The present invention is based on a new concept for the designing of sorbent materials which may be used in a laboratory scale as well as in the environmental area and other large scale operations.

According to the present invention there is provided a composite sorbent material in which a porous polymer gel material is used as a sorbent component. Preferably, the porous polymer gel material is a macroporous cryogel material.

Macroporous cryogel materials and their preparation are disclosed, e.g. by WO 03/031014 A1 and WO 03/041830 A2. However, due to their macroporosity particles or other shaped bodies of these materials will be most sensitive to abrasion when a suspension thereof is subjected to stirring.

In accordance with the present invention the degradation of such macroporous cryogel materials by abrasion is considerably reduced by surrounding such a material by a protective housing made of a dense insoluble material as a another component of the composite sorbent material.

Thus, according to one aspect of the present invention there is provided a composite sorbent material comprising

A) a housing of a dense insoluble material, and

B) a porous monolithic polymer gel housed within said housing,

said housing being provided with openings therein for fluid communication between said porous monolithic polymer gel and the surroundings, and

said porous monolithic polymer gel occupying the inner space of said housing and being kept on place within said housing by mechanical means.

According to another aspect of the present invention there is provided a number of processes for the preparation of a composite sorbent material according to the present invention said processes having the common feature of forming a porous gel material in the form of a monolith within a housing of a dense insoluble material having openings therein and being provided with mechanical means keeping the porous gel prepared on place. For instance, the porous gel may be prepared by polymerization of a solution of monomers or cross-linking a polymer in solution inside the housing of a dense, insoluble material.

According to a further aspect of the invention there is provided the use of the composite sorbent material according to the invention for the removal of pollutants from gases or liquids or for the enrichment or separation of molecules that bind to structures in the gels. Further uses of the composite sorbent material according to the invention will become evident from the detailed description that follows.

According to a still further aspect of the invention there is provided a method of culturing mammalian cells by continuously bringing a support material to which said mammalian cells are attached in contact with said mammalian cells in which method a composite sorbent material according to the invention is used as said support material and the mammalian cells are accommodated in the pores of said composite sorbent material.

### BRIEF DESCRIPTION OF THE DRAWINGS

FIG. 1 illustrates urokinase production by human kidney cell line HT 1080 when grown continuously on a 100 ml column packed with a composite sorbent material according to the invention.

FIG. 2 illustrates urokinase production by human colon carcinoma cell line HCT 116 when grown continuously on a 100 ml column packed with a composite sorbent material according to the invention.

FIG. 3 illustrates production and capture of urokinase secreted by human kidney cell line HT 1080. The cells were grown on a composite sorbent material according to the invention and the urokinase produced was captured on another composite sorbent material according to the invention.

FIG. 4 is a SDS-PAGE for separation of urokinase from HT 1080 cell culture using a IMAC (Immobilized Metal Affinity Chromatography) capture column packed with composite sorbent materials according to the invention.

### DETAILED DESCRIPTION OF THE INVENTION

According to the above mentioned first aspect of the invention there is provided a composite sorbent material comprising

A) a housing of a dense insoluble material, and

B) a porous monolithic polymer gel housed within said housing,

said housing being provided with openings therein for fluid communication between said porous monolithic polymer gel and the surroundings, and

said porous monolithic polymer gel occupying the inner space of said housing and being kept on place within said housing by mechanical means.

The material to be used for said housing may be varied within wide limits as long as it is dense and insoluble in liquids and may for instance be selected from metals and plastics materials.

The housing is provided with openings allowing fluid communication between the porous monolithic polymer gel and the surroundings and mechanical means in order to keep the porous monolithic polymer gel on place therein.

According to one embodiment of the composite sorbent material of the invention said mechanical means comprises irregularities such as protrusions on the internal surface of the housing.

According to another embodiment of the composite sorbent material of the invention said housing is provided with a plurality of openings distributed over the surface thereof, said openings functioning as said mechanical means by parts of said porous monolithic polymer gel extending thereinto.

The housing may be of different shapes. Thus, according to one embodiment of the composite material of the invention, the housing is substantially in the shape of an open-ended cylinder and the mechanical means comprises a lattice of bars defining a plurality of open-ended duct-shaped spaces within said housing and extending in a direction from one of the open ends of the cylinder to the other.

Products of this type which are called Kaldness carriers (Odegaard et al, 2000) are found on the marked. They are made of a plastics materials and are shaped substantially like a cylinder (length 7 mm and diameter 10 mm) with a cross inside the cylinder and fins on the outside.

However, the shape and the number and the arrangement of the openings are of subordinate importance as long as there is an effective fluid communication between the porous monolithic polymer gel and the surroundings outside the housing.

The porous polymer gel to be used as a component of the composite sorbent material according to the invention is preferably of a macroporous structure, most preferably a macroporous cryogel.

The term “cryogel” as used here and in the claims denotes a polymer gel which has been prepared by gelation at a temperature below the freezing point of the solvent used in the preparation.

According to one preferred embodiment of the composite sorbent material according to the invention the porous polymer gel housed within the housing is a cryogel which comprises a product obtained by polymerizing an aqueous solution of at least one water-soluble monomer selected from the group consisting of:

N-substituted and non-substituted (meth)acrylamides;

N-alkyl substituted N-vinylamides;

hydroxyalkyl(meth)acrylates;

vinylacetate;

alkylethers of vinyl alcohols;

ring-substituted styrene derivatives;

vinyl monomers;

(meth)acrylic acid and salts thereof;

silic acids; and

monomers capable of forming polymers via polycondensation;

under freezing at a temperature below the aqueous solvent crystallization point, at which solvent in the system is partially frozen with the dissolved substances concentrated in the non-frozen fraction of solvent to the formation of a cryogel.

Details on the preparation of cryogels of this type are e.g. found in WO 03/041830 A2 the disclosure of which is incorporated by reference herein in its entirety.

By applying the general teachings of WO 03/41830 A2 about the preparation of cryogels on the present inventions, the present invention provides, according to the second aspect thereof, a process for the preparation of a composite sorbent material according to the invention, which process comprises polymerizing an aqueous solution of at least one water-soluble polymer selected from the group consisting of:

N-substituted and non-substituted (meth)acrylamides;

N-alkyl substituted N-vinylamides;

hydroxyalkyl(meth)acrylates;

vinylacetate;

alkylethers of vinyl alcohols;

ring-substituted styrene derivatives;

vinyl monomers;

(meth)acrylic acid and salts thereof;

silic acids; and

monomers capable of forming polymers via polycondensation;

under freezing at a temperature below the aqueous solvent crystallization point, at which solvent in the system is partially frozen with the dissolved substances concentrated in the non-frozen fraction of solvent to the formation of a cryogel within a housing of a solid, dense material having two opposite open ends and being provided with mechanical means keeping the cryogel thus prepared on place.

Preferred monomers for use in the preparation of a cryogel for use in the present invention are selected from the group consisting of N-substituted and non-substituted (meth)acrylamides, hydroxyalkyl(meth)acrylates and (meth)acrylic acid and salts thereof. The monomers are most preferably used in combinations of at least two thereof.

According to the best mode contemplated at present a cryogel for use in the present invention is prepared by using a combination of acrylamide, N,N-methylene-bis-acrylamide and N,N,N′,N′-tetramethylenediamine as the monomers.

The resistance of the cryogels to shear-forces is increased by increasing the total concentration of the monomers in the solution from which they are prepared. On the other hand the porosity of the cryogels will be reduced by increasing said concentration. A proper balance between porosity and resistance to shear-forces may easily be established in each specific case by means of a series of experiments wherein the concentration of the total monomers is varied. The appropriate range for the total concentration of the monomers in the solution will vary with the specific system used. Thus, for instance, the total concentration of the monomers of the system acrylamide, N,N-methylene-bis-acrylamide and N,N,N′,N′-tetramethylenediamine will generally be within the range of from 2 to 20% (w/v), preferably from 4 to 10% (w/v) calculated on the reaction solution from which cryogels are prepared.

Initiator systems conventionally used in connection with the polymerization of monomers or combination of monomers of the groups specified above are used when necessary or desired.

The solvent or solvent system used for cryogelation is selected from the group consisting of water and mixtures of water and water-miscible organic solvents.

Preferably the solvent to be used for the cryogelation is water alone but water in admixture with a minor amount of one or more water-miscible organic solvents, such as methanol, ethanol, dioxane, acetone, dimethyl sulfoxide, N,N-dimethylformamide and acetonitrile may also be contemplated.

The temperature to which freezing or chilling is carried out depends on the crystallization point of the solvent or solvent system used in each specific case. Said temperature should generally be at least 5° C. below freezing point of the solvent or solvent system in order to keep the crystallization time down. For instance, in case of water as the solvent, freezing is generally carried out to a temperature within the range of from −5° C. to −40° C., preferably from −10° C. to −30° C.

According to one mode of practising this process according to the invention a number of housings of a kind as defined above are loaded into a vessel which is then filled with a reaction solution comprising the monomers to be polymerized or the polymers to be cross-linked to a level to cover said housings where after said vessel is subjected to a temperature below the crystallisation point of the solvent or solvent system used in the polymerisation or polymer cross-linking at which solvent is partially frozen with the dissolved substances concentrated in the non-frozen fraction of solvent. After the formation of the cryogel the content of the vessel is cut frozen to separate the housings with cryogel therein from each other. The composite sorbent material according to the invention thus obtained is then subjected to thawing, e.g. at room temperature, followed by intensive washing with water.

According to another embodiment of the composite sorbent material according to the invention the porous polymer gel housed within the housing is a cryogel which comprises a product prepared by cryogelation of at least one synthetic or natural polymer in the presence of a chautropic agent and, if necessary or desired, a cross-linking agent.

Poly(vinyl alcohol) is a preferred representative of said at least one synthetic polymer to be used in this embodiment of the composite sorbent material according to the invention. Most preferably the poly(vinyl alcohol) is used in a cross-linked form. Examples of cross-linking agents contemplated for use in this connection are epichlorohydrin, divinyl sulfone, glutaraldehyde and di- and triglycidyl compounds, preferably glutaraldehyde.

Said at least one natural polymer to be used in this embodiment of the composite sorbent material according to the invention may be at least one member selected from the group consisting of polysaccharides and proteins.

Examples of polysaccharides contemplated for use in this connection are agarose, agar, carrageenans, starches, cellulose and their respective derivatives.

An example of a protein contemplated for use in this connection is chitosan.

The present invention further provides a process for the preparation of a composite sorbent material according to this embodiment of said material according to which process at least one substance selected from the group consisting of synthetic and natural polymers are subjected to cryogelation in the presence of a chaotropic agent and if necessary, a cross-linking agent within a housing of a dense, insoluble material having openings therein and being provided with mechanical means keeping the monolithic cryogel thus prepared on place.

Details on how to prepare cryogels from synthetic and natural polymers are e.g. found in WO 03/031014 A1, the disclosure of which is incorporated by reference herein in its entirety.

In brief, a solution of said at least one synthetic or natural polymer in water or a mixture of water and a water-miscible organic solvent in cooled in the presence of a chaotropic agent to a temperature at which solvent in the system is partially frozen with the dissolved substance or substances concentrated in the non-frozen fraction of the solvent.

A cross-linking agent may be present during the cooling or cross-linking may be performed after the formation of the cryogel.

The chaotropic agent used in this process may be selected from the group consisting of urea, alkyl ureas, guanidine chloride, LiCl, KSCN, NaSCN, acids and bases and mixture thereof.

For instance, as an example of this embodiment of the process according to the invention a composite sorbent material having agarose as its sorbent component may be prepared by filling an alkaline aqueous solution of agarose into a housing as defined previously and frozen at a temperature within a range below −10° C. to enable the formation of cryogel of agarose.

As another example of this embodiment of the process according to the invention a composite sorbent material having cross-linked poly(vinyl alcohol) as its sorbent component may be prepared by filling an acid aqueous reaction mixture of poly(vinyl alcohol) and glutaraldehyde into a housing as defined previously and freezing is at a temperature within a range below 0° C. to enable the formation of a cryogel of cross-linked poly(vinyl alcohol).

As a further example of this embodiment of the process according to the invention a composite sorbent material having cross-linked chitosan as its sorbent component may be prepared by filling an aqueous solution of chitosan buffered to a pH within the range of from 4.5 to 5.6 into a housing as defined previously and cooling to a temperature within a range below 0° C. until said solution is frozen where after the chitosan is cross-linked by means of a solution of glutaraldehyde in ethanol at a temperature within a range below 0° C. to enable the formation of a cryogel of cross-linked chitosan.

The present invention also comprises a process which in addition to the steps disclosed in relation to the process embodiments discussed above comprises the additional step of adding an aqueous solution of a gel-forming compound, either alone or mixed with fillers or cells, to a composite sorbent material prepared according to said embodiments and cooling the mixture obtained to a temperature within a range below 0° C. until said solution is frozen, whereafter the double frozen cryogel thus formed is thawed.

According to another aspect of the invention the properties of the composite sorbent material may be modified by modifying the sorbent component thereof.

Thus, the present invention also provides a composite sorbent material according to the invention wherein porous polymer gel of the sorbent component thereof has been modified by introducing a member selected from the group consisting of ligands, charged groups and hydrophobic groups thereinto.

Ligands to be used in this embodiment of the composite sorbent material according to the invention may be selected from the group consisting peptides, metal chelates, sugar derivatives, boronate derivatives, enzyme substrates and their analogues, enzyme inhibitors and their analogues, protein inhibitors, antibodies and their fragments and thiol containing substances.

The present invention also provides a composite sorbent material according to the invention wherein the porous polymer gel of the sorbent component thereof has been modified by introducing a filler selected from the group consisting of metals, metal oxides, ion exchange particles, hydrophobic adsorbents, affinity adsorbents and molecularly imprinted polymers in the form of particles.

As a further modification of the composite sorbent material according to the invention the sorbent component thereof is a cryogel prepared by means of polymerization in the presence of a template molecule, thereby forming a molecularly imprinted polymer gel.

According to a further aspect of the present invention there is provided, according to one embodiment of said aspect, the use of the composite sorbent material according to the invention for the removal of pollutants from gases or liquids.

According to another embodiment of said further aspect there is provided the use of the composite sorbent material according to the invention for the enrichment or separation of molecules that bind to structure in the porous polymer gel.

According to a further embodiment of said further aspect there is provided the use of a composite material according to the invention that is loaded with a biocatalyst for bioconversion of substances in a medium to be contacted with the composite sorbent material.

The biocatalyst to be used in this embodiment may be selected from the group consisting of enzymes (proteases, amylases, lipases, etc) and cells [prokaryotic cells (as, for example, Lactobacillus) as well as eukaryotic cells (for example baker's yeast cells (Saccharomyces cerevisiae) and mammalian cells (different cell lines such as, e.g. CD34+KG-1 human tumour cells, mouse embryonic fibroblast cell line 3T3-L1, hybridoma cell line MD2139 etc.)]

According to a still further embodiment of said further aspect of the invention there is provided the use of the composite sorbent material according to the invention for the immobilization of cells.

According to a further embodiment of said further aspect of the present invention there is provided the use of the composite sorbent material according to the invention in cell culture.

According to a preferred way of practising this embodiment a suspension of cells is applied to a column filled with a composite sorbent material according to the invention, the column is then incubated for a period sufficient to bind the cells sufficiently to the composite sorbent material whereafter a culture medium is circulated through the column.

According to a still further embodiment of the present invention there is provided a method of culturing mammalian cells by continuously bringing a liquid culture medium in contact with said mammalian cells in a bioreactor containing a support material to which said mammalian cells are attached, wherein in a composite sorbent material according to the invention is used as said support material, said mammalian cells being accommodated in the pores of said composite sorbent material.

According to a preferred embodiment of this aspect of the invention liquid culture medium from a media reservoir is continuously circulated through the bioreactor.

In this preferred embodiment culture medium leaving the bioreactor is preferably passed through an affinity absorber comprising an appropriate composite sorbent material according to the invention in order to capture target products released from the bioreactor before the culture medium being returned to the media reservoir.

The term “a composite sorbent material” as used here and in the claims in connection with the use thereof is intended to include the use of one single unit of a housing and a porous polymer gel therein as well as the use of a plurality of such units in combination.

The invention will now be further illustrated by means of a number of non-limitative examples.

### EXAMPLE 1

### Preparation of a Macroporous Gel from Low Molecular Weight Precursors in a Plastic Housing

a) Preparation of a Macroporous Gel from a Reaction Mixture Having a Total Monomer Concentration of 5.0% (w/v).

Acrylamide (AAm) (3.19 g), N,N-methylene-bis-acrylamide (MBAAm) (1.17 g) and allylglycidylether (AGE) (1.0 ml) were dissolved in deionized water (total volume of the reaction mixture: 103 ml, final monomer concentration: 5.0%). The reaction mixture was degassed in vacuo (water pump aspirator) for 10 min to eliminate soluble oxygen. After cooling of the reaction mixture for 20 min a free radical polymerization was initiated by adding N,N,N′,N′-tetramethyleneethylenediamine (TEMED) (80 μl) and ammoniumpersulfate (APS) (64 mg). A glass column having an inner diameter of 13 mm and a removable bottom was filled with Kaldness carriers (K1) (open-ended cylinder-like plastic bodies from Kaldness miljotechnologi AS, Tönsberg, Norway, having a length of 7 mm and a diameter of 10 mm with a cross inside the cylinder and fins on the outside) and then with the reaction mixture. In order to prevent the Kaldness carriers (in the following alternatively called “plastic carriers” or simply “carriers”) from floating on top on the reaction solution (density of said plastic carriers 0.92-0.96 g/cm³) a lid was put on top of the carriers. The glass column, filled with the reaction mixture/plastic carriers, was set inside a low temperature thermostat (Lauda-RK20KP from LAUDA Dr. R. WOBSER GmbH & Co. KG, Lauda-Königshofen, Germany) for freezing at −12° C. After kept frozen overnight the plastic carriers with polyacrylamide containing epoxy groups formed inside the plastic carriers were separated from each other by removing the removable bottom of the column, pressing out the frozen mass and cutting loose each unit of the composite sorbent material using a sharp knife or a razor blade and then thawed at room temperature by intensive washing with water.

Due to the presence of the cross of bars within the Kaldness carriers there is formed a lattice defining a plurality of open-ended duct-shaped spaces therein extending in a direction from one of the open ends of the cylinder-like Kaldness carrier to the other which duct-shaped spaces as a consequence of the polymerization reaction above became filled with a macroporous polymer of acrylamide exhibiting epoxy groups. A composite sorbent material according to the present invention comprising a Kaldness carrier with a macroporous cryogel within the duct-shaped spaces therein as described above will in the following be called a “minicolumn”.

b) Preparation of a Macroporous Gel from a Reaction Mixture Having a Total Monomer Concentration of 7.0% (w/v)

AAm (4.53 g), MBAAm (1.64 g) and AGE (1.42 ml) were dissolved in deionized water (total volume of the reaction mixture: 107 ml; final monomer concentration 7.0% (w/v). The reaction mixture was degassed and cooled as set forth in Section a) above whereafter free radical polymerization was initiated by adding TEMED (113 μl) and APS (90 mg). Then the further procedure was as set forth in Section a) above.

c) Preparation of a Macroporous Gel from a Reaction Mixture Having a Total Monomer Concentration of 11.5% (w/v)

AAm (7.35 g), MBAAm (2.69 g) and AGE (2.31 ml) were dissolved in deionized water (total volume of the reaction mixture: 107 ml; final monomer concentration 11.5% (w/v). The reaction mixture was degassed and cooled as set forth in Section a) above whereafter free radical polymerization was initiated by adding TEMED (184 μl) and APS (147 mg). Then the further procedure was as set forth in Section a) above.

### EXAMPLE 2

### Preparation of Minicolumns Comprising Macroporous Polyacrylamide Gel Modified by Iminodiacetic Acid

The starting material used in this experiment were minicolumns prepared according to example 1 using the three different concentrations of total monomers as indicated in said example, namely 5.0, 7.0 and 11.5% (w/v), of the reaction solution.

The minicolumns comprising epoxy-modified polyacrylamide were washed with 0.5 M Na₂CO₃ solution and placed into a glass bottle. Iminodiacetic acid (0.5 M in 1 M Na₂CO₃) was added in an amount to cover the minicolumns in the bottle. The reaction was carried out at stirring on rocking table for 24 h at room temperature. Finally, the minicolumns were washed with water until neutral pH.

Minicolumns comprising macroporous polyacrylamide gel modified by iminodiacetic acid were obtained.

### EXAMPLE 3

### Preparation of Minicolumns Comprising Macroporous Polyacrylamide Gel Containing Ion-Exchange Groups

Twenty (20) minicolumns of polyacrylamide (pAAm) containing epoxgroups prepared according to example 1 using a total monomer concentration of 5.0% (w/v) were washed with 0.1 M sodium carbonate buffer pH 9.5. Then a solution of N,N-dimethyltrimethylenediamine (0.3 M in 0.1 M sodium carbonate buffer, pH 9.5, 50 ml) was applied to the minicolumns at stirring on rocking table for 24 hrs. The minicolumns exhibiting anion-exchange groups in the form of tertiary amino (—N(CH₂)₂) groups thus obtained were washed with water until neutral.

### EXAMPLE 4

### Preparation of Minicolumns Comprising Macroporous Chitosan-Based Gel

A viscous aqueous chitosan solution (2%, w/v) was diluted with 0.1 M Na-acetate buffer, pH 5.6 at a final concentration of 0.5% (w/v). Then, the chitosan solution was poured into a plastic syringe (i.d. 12 mm) filled with 6 Kaldness carriers and was frozen at −20° C. After kept frozen for 1 h, the frozen minicolumns were removed from the syringe, cut frozen (to separate the minicolumns from each other), and transferred into a cross-linker solution (2.5%, v/v glutaraldehyde (GA) in EtOH, GA/EtOH, 5/95, v/v), 15 ml, cooled to −20° C. and was kept in the cross-linker solution at −12° C. overnight. The colour of the frozen monolith formed became yellow indicating the formation of the Schiff bases during the storage in the GA/EtOH solution in frozen state. Then the frozen monolith in the GA solution was thawed at room temperature for 4 h while additional cross-linking of the thawed cryogel matrix proceeded. The thawed cryogel minicolumns were washed by gradual replacement of ethanol to water until neutral pH. After the applying of the sodium borohydrate solution (0.1 M) in 0.1 M Na-carbonate buffer for 3 h, the colour of the chitosan cryogel changed from the brown to light yellow due to reduction of the Schiff bases. Finally the chitosan minicolumns were washed with water. The minicolumns obtained were designated “0.5-Cts-PC”.

For the preparation of chitosan minicolumns from 1% chitosan solution, the aqueous chitosan solution (2%, w/v) was diluted with 0.1 M Na-acetate buffer, pH 5.6 at a final concentration of 1.0% (w/v). Then, the preparation of chitosan minicolumns (“1-Cts-PC”) was performed as described above.

Chitosan minicolumns with ordinary chitosan gels (i.e. at room temperature) (“1 gel-Cts-PC”)were prepared for comparison with 1-Cts-PC as follows: an aqueous chitosan solution (2%, w/v) was diluted with 0.1 M Na-acetate buffer, pH 5.6 at a final concentration of 1.0% (w/v). The cross-linker, GA, was added to the chitosan solution till final concentration of 0.1% (v/v) and the reaction mixture was quickly poured into a plastic syringe (i.d. 12 mm) filled with 6 Kaldness carriers and was kept at room temperature overnight. The colour of the formed rigid chitosan gel became yellow indicating the formation of the Schiff bases. Then the formed rigid chitosan gel was removed from the syringe and the minicolumns were cut (to separate the minicolumns from each other), and transferred into a glass bottle with the solution of sodium borohydrate (0.1 M) in 0.1 M Na-carbonate buffer (to cover the minicolumns with the solution). The reaction was carried out at stirring on rocking table for 3 h. The color of the chitosan gel changed from the brown to light yellow due to reduction of the Schiff bases. Finally the chitosan minicolumns were washed with water. As the prepared 1 gel-Cts-PC ordinary gel was rigid (compared to 1-Cts-PC cryogel, which is elastic and spongy-like), the weight loss for 1 gel-Cts-PC was much higher (46%) than for 1-Cts-PC (26%) in a study on the mechanical stability (vide Example 14 below).

### EXAMPLE 5

### Preparation of Minicolumns Comprising Macroporous Agarose-Based Gel

An aqueous solution of agarose (2.0%, w/v) was prepared by stirring at elevated temperature (90° C.). The agarose solution was cooled down to 60-65° C. and the pH thereof was adjusted by means of concentrated NaOH (5M) to an alkali concentration of 0.1 M (pH 13.0). Then the warm alkaline solution of agarose was poured into a plastic syringe (i.d. 12 mm filled with 6 Kaldness carriers) and frozen at −35° C. The samples were kept frozen at −35° C. for 1 hr and at −12° C. overnight. The frozen samples were thawed at a thawing rate of 0.06° C./min and washed with water until neutral pH.

### EXAMPLE 6

### Preparation of Minicolumns Comprising Macroporous Blue Coloured Agarose-Based Gel

Example 5 was repeated but a blue dye (Cibacron Blue, 40 mg) was added to the hot agarose solution before adjustment of pH with concentrated NaOH.

### EXAMPLE 7

### Preparation of Minicolumns Comprising Macroporous Gel Based on a Poly(Vinyl Alcohol) (PVA)

PVA (PVA of grade 8-88, MW 67000 g/mol from Mowiol; degree of saponification 88%) was dissolved in water (5%, w/v) by stirring at elevated temperature (90° C.). After cooling the PVA solution to room temperature, the pH of the solution was adjusted to 1.0-1.2 with 5 M HCl and the solution was cooled in an ice bath for 30 min. Glutaraldehyde (cross-linking agent, final concentration 1.0% w/v) was added and the reaction mixture was stirred for 1 min. The solution was poured into plastic syringes (i.d. 12 mm filled with 6 Kaldness carriers) and was frozen at −18° C. After kept frozen at −18° C. overnight, the frozen minicolumns were defrosted and washed with water until neutral pH.

### EXAMPLE 8

### Preparation of Minicolumns Comprising Macroporous Gel Based on Carboxyl-Modified PVA

Minicolumns comprising macroporous gel based on PVA prepared according to example 7 were reacted with 0.5 M chloroacetic acid in 0.1 M sodium carbonate buffer, pH 8.0 at stirring at 100 rpm for 16 hrs at room temperature. The minicolumns comprising macroporous cross-linked PVA-gel exhibiting carboxy groups thus prepared were washed with water until neutral pH.

### EXAMPLE 9

### Preparation of Minicolumns Comprising Macroporous Acrylamide Gel with Immobilized Enzyme (Trypsin)

Trypsin solution (5 mg/ml in 0.1 M Na-phosphate buffer, pH 8.0) was applied at stirring at 100 rpm for 24 h to minicolumns of polyacrylamide containing epoxi groups prepared according to example 1 using a total monomer concentration of 7.0% (w/v). After blocking of unreacted epoxy groups with 0.1 M ethanolamine for 3 h, the minicolumns with immobilized enzyme were washed with water until neutral pH and were kept in 0.1M Na-phosphate buffer, pH 7.4 at 4° C.

A spectrophotometric assay was used to check the activity of the immobilized enzyme, implying the cleavage of the specific substrate N-benzoyl-DL-arginine-4-nitoanilide (BAPNA) at 405 nm. For this purpose the determined amount of the minicolumns (12 pieces) with immobilized enzyme (biocatalyst) containing approximately 0.6 mg of the protein was suspended in 30 mL of 0.05 M Tris-HCl buffer solution, pH 8.0 and thermostated at 37° C. for 15 min. The BAPNA substrate (200 μl of 1 mM solution in MeCN) was added, and the reaction mixture was stirred at thermostated reactor at 37° C. at stirring at 100 rpm. The absorbance of the solution at 405 nm was monitored with time. The control hydrolysis of the BAPNA substrate at the same conditions was detected as well. Molar absorptivity for p-nitroaniline liberated was taken equal to 9620 M⁻¹ cm⁻¹.

### EXAMPLE 10

### Preparation of Minicolumns Comprising Acrylamide Gel with Immobilized Yeast Cells

Different amounts of yeast cells (Saccharomyces cerevisiae, purchased in the form of blocks from a local supplier) were mixed with a monomer solution of acrylamide (AAm) and N,N-methylene-bis-acrylamide (MBAAm) (AAm+MBAAm 8% w/v), AAm/MBAAm 10/1, mol/mol) to concentrations of 0.1, 0.5 and 1.0% (w/v), respectively. After the addition of an initiating system of ammoniumpersulfate (APS) and N,N,N′,N′-tetramethyleneethylenediamine (TEMED) (1% w/v of the total monomer concentration (AAm+MBAAm)), the reaction mixture was poured into syringes filled with Kaldness carriers and frozen at −20° C. for 1 h. After having been kept frozen at −12° C. overnight, the prepared minicolumns with immobilized yeast cells were removed from the syringe, cut frozen to separate the minicolumns from each other and thawed at room temperature. The minicolumns were then washed with water under intensive stirring and then with 20 mM Tris-HCl buffer, pH 7.2. The washed minicolumns were dried in an oven at 30 and 60° C. overnight. The dried minicolumns were placed in a glass bottle and reswollen in water. The viability of the immobilized yeast cells just prepared and after drying and re-swelling was estimated as follows: the minicolumns were placed in a glass bottle and equilibrated with a buffer composed of 20 mM Tris-HCl buffer pH 7.4 with 0.95 mM CaCl₂, 5.56 mM KCl, 137 mM NaCl, 0.8 mM KH₂PO₄ and 0.41 mM NaHCO₃ at stirring for 30 min. Then, the immobilized cells minicolumns were equilibrated with glucose solution (50 mM), containing 0.02% neutral red, by passing 1 ml of the glucose solution through each minicolumn. Finally, the glucose solution (2 ml) was added to the glass bottles with the minicolumns and the glass bottles were stirred on rocking table for 4 h. After decanting the supernatant, the minicolumns were additionally washed with buffer (2 ml) for 30 min. The absorbencies at 528 nm and the pH value were checked for the pooled supernatants.

The absorbance at 528 nm was increased proportionally for the minicolumns with increased amount of immobilized yeast cells.

The viability test for the immobilized cells carried out on minicolumns having a cell load of 0.5% before and after drying at 30 and 60° C. showed 92 and 77% of remained activity of the immobilized cells at these temperatures, respectively.

SEM images for minicolumns with a cell loading of 1% showed the yeast cells incorporated into the polymeric network.

### EXAMPLE 11

### Preparation of Minicolumns Comprising Acrylamide Gel with Immobilized E. Coli Cells

E. coli cells were immobilized into minicolumns containing macroporous gel and being prepared analogous to Example 1 from a reaction solution of 6% monomer concentration. A sequential freezing approach was used as follows: The macroporous gel microcolumns were dried in an oven at 60° C. overnight. A suspension of E. coli cells (1 wt %) in poly(vinyl alcohol) (PVA) solution (6.5 wt %, PVA with grade 20-98 from Mowiol, MW 125000 g/mol) was prepared under gentle stirring in ice bath. The dried microcolumns (20 microcolumns) were poured into the suspension of E. coli cells (30 ml) and were mixed under stirring in an ice bath for 30 min. After decanting the cell suspension, the microcolumns filled with the cell suspension were placed into plastic syringes (12 mm i.d.) and were frozen at −20° C. for 1 h. After kept frozen at −12° C. overnight, the frozen minicolumns were removed from the syringe, cut frozen (to separate the minicolumns from each other), placed again inside the plastic syringe and thawed at a thawing rate of 0.04° C./min inside a low temperature thermostat LAUDA-RK20KP with selected thawing program. After that the minicolumns were washed with water until zero absorbance at 620 nm (no cells were leaching out). The washed minicolumns were dried in oven at 30° C. for 24 h followed by re-swelling in water and then in buffer.

The formed macroporous gels can be referred as monolith cryogels with interpenetrating macroporous network (IPMN) as they were formed from two polymeric systems, PVA and pAAm (PVA/pAAm-IPMN-cryogels) Specifically, in this case E. coli-PVA/pAAm-IPMN gels were formed inside the plastic carriers

Before a viability test, the immobilized E. coli cells were re-activated by incubation of the EcPVA-pAAm-minicolumns above in LB medium at stirring at 120 rpm at 37° C. for 16 h. The cell viability control was performed as follows: freshly prepared solutions of tetrazolium salt XTT (SIGMA, product number X4251) (1 mg/ml) and the electron mediator reagent Menadione (1.72 mg/ml dimethyl sulfoxide) were mixed with LB medium (in the proportions 0.2 ml menadione/1 ml XTT/1.2 ml LB medium) immediately before use. The Ec/PVA-pAAm-particles were equilibrated first with 20 mM HEPES, pH 7.2 with 0.2 M NaCl buffer and then with freshly prepared mixture (2 ml of the mixture was passed through each MG-particle). Two ml of the mixture was applied to the bottles with determined amount of Ec/PVA-pAAm-particles (3 st) equilibrated with 20 mM HEPES, pH 7.2 with 0.2 M NaCl) and were incubated at 37° C. for 2 h. The supernatant was decanted and the particles were washed with 2 ml of 20 mM HEPES, pH 7.2 with 0.2 M NaCl for 40 min at stirring on rocking table. The formazan product was measured at 470 nm in the pooled supernatants fractions.

The viability test showed 32% of retained activity for the E. coli cells in the EcPVA-pAAm-minicolumns.

### EXAMPLE 12

### Preparation of Minicolumns Comprising Acrylamide and a Filler

Agarose based minicolumns were prepared as described in Example 5 except that Sepharose-6B (cross-linked agarose gel from Amersham Pharmacia Biotech AB, Uppsala, Sweden (now GE Healthcare) was added as a filler to the hot agarose solution to a final concentration of 20% (w/v). IDA functionality was introduced as follows: 20 minicolumns containing filler were mixed with a suspension of epichlorohydrin (10%, v/v) in 1.0 M NaOH at stirring at 100 rpm overnight. After washing with water until neutral pH, the epoxy-activated minicolumns were treated first with 0.5 M Na₂CO₃ for 20 min and the IDA ligand (0.5 M in 1M Na₂CO₃, pH 10.0) was applied to the minicolumns at stirring at 100 rpm. overnight. Finally, the IDA-minicolumns were washed with water until neutral pH.

### EXAMPLE 13

### Preparation of Minicolumns Comprising PVA-Based Macroporous Gels with Molecular Imprinted (MIP) Beads with β-Extradiol used as a Template

First MIP-beads were prepared according to the following procedure: β-estradiol (272 mg) was dissolved in 8 ml of acetonitrile in a dried glass vial (30 ml). As a functional monomer 4.27 mL of 4-vinylpyridine and 50 mg of azobisisobutyronitrile were added. All components were gently mixed and were sonicated to dissolve possibly un-dissolved β-estradiol. The reaction mixture was purged with nitrogen for 5 minutes. The test tube was sealed tightened and heated in a water bath (65° C.) for at least 20 h. After that, the polymer monolith was withdrawn from the water bath and the tube was broken for gathering the monolith particles. The particles were grounded manually in a mortar and passed through three micron test sieves (to get the MIP-particles with size in the range of 38-106 μm) and washed with methanol. The flask, containing the fine MIP-particles in methanol was kept under the fume hood until methanol was completely evaporated. The dried MIP-particles were washed separately in a Soxhlet extractor with methanol for 20 h and dried afterwards under a fume hood for 24 h.

The prepared MIP-beads (bead size 38-106 μm), 300 mg, were mixed with 4.8 ml of a PVA solution (PVA with grade 8-88, MW 67000 g/mol from Mowiol; degree of saponification 88%) of 5.4% concentration with pH 1.0. The reaction mixture was cooled in ice bath for 5 min. The cross-linker GA (final concentration 1.25% w/v) was added and the reaction mixture was stirred for 1 min. The solution was poured into the plastic syringes (i.d. 12 mm filled with 6 Kaldness carriers) and was frozen at −20° C. After kept frozen at −12° C. overnight, the frozen macroporous gel-particles were defrosted and washed with water until neutral pH. The treatment with ethanolamine to (0.2 M in 0.1 M Na-phosphate buffer, pH 8.2) was performed at stirring on rocking table for 5 h. After washing with water, the minicolumns were treated with sodium borohydrate solution (0.2 M in 0.1 M Na-carbonate buffer, pH 9.5) for 3 h to reduce formed Schiff bases. Finally, the composite MIP/PVA-minicolumns were washed with water until neutral pH. In a control experiment, the PVA-particles were formed as described above except that no MIP-particles were added to the PVA solution.

Analysis of MIP/PVA minicolumns was performed as follows: the MIP/PVA particles (6 minicolumns) and PVA-particles as a control (6 particles) and empty Kaldness carriers (as a control) were transferred into a beaker and washed for 2 h with 100 mL of methanol:acetic acid (4:1 v/v) to remove any traces of β-estradiol. Then the washed particles were transferred into the beaker containing 100 ml of β-estradiol solution (0.5 mg/L) and were stirred for 24 h at 120 rpm. Aliquot of 1 ml were taken after 2, 18 and 24 h and were analysed with HPLC. Then, the particles were transferred to a beaker containing 100 ml of elution agent (methanol:acetic acid (4:1 v/v)) and were stirred at 120 rpm for 18 hours. The 1 ml aliquots were taken after 2, 18 and 24 h and were analysed with HPLC.

The results are reported in Table 1 below.

TABLE 1
Removal of β-estradiol from water using MIP/PVA particles

| | | PVA- | Empty plastic |
|---|---|---|---|
| | MIP/PVA- | minicolumns | carriers |
| | minicolumns | (control) | (control) |
| Captured | 100.0 | 72.1 | 0 |
| β-estradiol | | | |
| (%)* | | | |
| Recovered | 105.0 | 103.0 | 0 |
| β-estradiol | | | |
| (%)** | | | |

*Applied β-estradiol was taken as 100%
**Absorbed β-estradiol was taken as 100%

From the table it is seen that all β-estradiol was captured from water by MIP/PVA-minicolumns while the control PVA-minicolumns non-specifically absorbed 72% and there was no non-specific sorption of β-estradiol with the empty plastic carriers.

SEM images MIP/PVA and PVA (control) showed the large size of interconnected pores in the MIP/PVA minicolumns with pieces of the incorporated MIP beads. SEM of PVA (control) showed the large size of interconnected pores with noticeable microporosity of pore walls in PVA. That, probably, was the reason for the non-specific capturing of the β-extradiol by PVA-minicolumns (control).

### EXAMPLE 14

### Study on the Mechanical Stability of pAAm Minicolumns

The mechanical stability of the pAAm minicolumns prepared according to Examples 1a to 1c was evaluated as ability of the cryogel to be held inside the plastic housing at stirring at 400 rpm. The long-term stirring at 400 rpm showed that the weight loss was less for the more dense gel matrix (i.e. for the minicolumns of Example 1c). More than 70% of the minicolumns of Example 1a was destroyed due to the intensive stirring at 400 rpm. About 12% and 19% of weight loss was observed for minicolumns of Example 1b and 1c, respectively, for the first 10 days of stirring at 400 rpm. Subsequent stirring did not resulted in a significant decrease in the gel weight showing the protective role of the plastic housing against shear forces coursed by intensive stirring. Comparison of some other minicolumns according to the invention prepared from different monomer/polymer precursors to withhold intensive stirring at 400 rpm are presented in Table 2 below.

TABLE 2
Weight loss for the different minicolumns
after stirring at 400 rpm for 24 h

| Sorbent: | Weight loss, %* |
|---|---|
| Example 2 (5% monomer) | 14 |
| Example 7 | 2 |
| Example 4 (1-Cts-PC) | 26 |
| Example 4 (1gel-Cts-PC) | 46 |
| Example 5 | 32 |
| Example 12 | 36 |

*After stirring at 400 rpm for 24 h

### EXAMPLE 15

### Batch-Mode Sorption of Cu(II)

Batch sorption experiments were performed by contacting 5.5 g (or 10 minicolumns) of IDA-pAAm minicolumns from Example 2 (monomer concentrations 7.0 and 11.5%) with 10 ml of a aqueous Cu(II) solution of different initial concentration (100 and 1000 mg/L) at stirring at 100 rpm. The remaining Cu(II) concentration in each sample after adsorption at different time intervals was determined by atomic absorption spectroscopy. The uptake of Cu(II)-ions was calculated using the equation: Uptake, %=[(Cₒ−Cₑ)/Cₒ)]×100, where Cₒ and Cₑ are the initial and equilibrium concentrations (mg/L). The adsorption of Cu(II) by the minicolumns reached equilibrium in less than 20 minutes for both types of minicolumns but somewhat slower for the 11.5% type. The adsorbed metal ions were readily stripped by EDTA or acid treatment and regenerated.

### EXAMPLE 16

### Batch Sorption of Lysozyme

Binding of lysozyme to the minicolumns with adsorbed Cu(II) prepared according to Example 15 was performed as follows: lysozyme solution (0.2 mg/ml), 10 ml, in 20 mM Tris-HCl, pH 7.0 was applied to 5.5 g of minicolumns from Example 15 (or 10 minicolumns) (7.0% monomer concentration) at stirring at 100 rpm for 1 h. The remaining protein concentration in each sample was determined spectrophotometrically at 280 nm. The lysozyme uptake was calculated using the equation: lysozyme uptake, %=[(Cₒ−Cₑ)/Cₒ)]×100, where Cₒ and Cₑ are the initial and equilibrium concentrations (mg/ml). The lysozyme uptake calculated in this way was found to be 13.4%.

### EXAMPLE 17

### Batch Sorption of Carboxy-Modified Latex Particles

Binding of carboxy-modified latex particles with diameter of 3 μm (these particles are composed primarily of polystyrene (95-99%) and a secondary acidic monomer, typically acrylic acid, is used to add carboxyl groups to the surface). (Data from the manufacturer: Seradyn, Indianapolis, USA) to the chitosan-based minicolumns was performed as follows: a dispersion of latex particles in 20 mM Tris-HCl, pH 7.0 (absorbance at 620 nm 0.18) was applied to 5.5 g of chitosan-based minicolumns prepared according to Example 4 (or 10 minicolumns) at stirring at 100 rpm. The remaining latex concentration in each sample after adsorption at different time intervals was determined spectrophotometrically at 620 nm. The latex particles uptake was calculated using the equation: latex uptake, %=[(Aₒ−Aₑ)/Aₒ)]×100, where Aₒ and Aₑ are the absorbance at 620 nm for initial and equilibrium solution (mg/ml). More than 74 and 56% of the latex particles were absorbed to the 1-Cts-PC and 0.5-Cts-PC, respectively, after 1 h of stirring at 100 rpm.

### EXAMPLE 18

### Batch Sorption of Yeast and E. Coli Cells

Binding of yeast and E. coli cells to ion-exchange minicolumns was performed as follows: a suspension of yeast and E. coli cells in 20 mM Tris-HCl, pH 7.0 (absorbance at 620 nm 0.08 and 0.1, accordingly) was applied to minicolumns prepared according to Example 3 at stirring at 100 rpm. More than 69 and 87% of Yeast and E. coli cell, respectively, were bound to the ion-exchange minicolumns bearing positive charges on the gel surface at these conditions (Example 3).

### EXAMPLE 19

### Packed-Bed Sorption Experiment

A (30×5.0 cm) scaled glass column, initially designed for expanded bed chromatography, was filled with minicolumns prepared according to Example 2 (7.0% monomer concentration) (bed volume 0.5 L). After fitting with the upper movable adaptor, the column was connected to a pump. The mobile phase (deionized water) was passed through the column upwards at a flow rate of about 60 cm/h. Copper solution was pumped through the column upwards at the given concentration at two different flow rates (5 and 20 ml/min, that corresponds to 15 and 60 cm/h). Samples (volume 250 ml) were collected and analyzed for Cu(II) content using atomic absorption spectroscopy. Absorbed Cu(II) ions were desorbed from the column with 0.1 M EDTA solution in up-down mode. The enrichment of Cu in the eluate was high and the solution from where Cu was enriched was depleated with regard to Cu content.

### EXAMPLE 20

### Preparation of the Gelatin-Cryogel Minicolumns for Cell Culture

The epoxy-containing supermacroporous monolithic cryogel of Example 1 (prepared from monomer concentration of 5%) was treated as follows. A 100 ml column packed with cryogel-containing minicolumns was washed first by passing 300 ml of water through the column followed by 0.5 M Na₂CO₃ (300 ml) at a flow rate of 2 ml/min. Ethylenediamine (0.5 M in 0.2 M Na₂CO₃; 300 ml) was then passed through the cryogel column for 4 h in recycle mode at flow rate of 1 ml/min. After washing with water until the pH was close to neutral, the column was washed with 300 ml of 0.1 M sodium phosphate buffer, pH 7.2. A solution of glutaraldehyde (5% v/v; 300 ml) in 0.1 M sodium phosphate buffer, pH 7.2, was passed through the column in recycle mode overnight at flow rate of 1 ml/min. The column was then washed with water and subsequently with 0.1 M sodium phosphate buffer, pH 7.2. The derivatized matrix with functional aldehyde groups was used for coupling of gelatin. A solution of gelatin from cold water fish skin (5 mg/ml; 300 ml) in 0.1 M sodium phosphate buffer, pH 7.2 was circulated through the column for 24 h at 4° C. Finally, the freshly prepared NaBH₄ solution (0.1 M in sodium carbonate buffer, pH 9.2; 300 ml) was applied to the column to reduce Schiff's base formed between the protein and the aldehyde-containing matrix.

The amount of gelatin immobilized on the acrylamide monolithic cryogel matrix was determined by the bicinchoninic acid (BCA) method (Smith, P. K.; Krohn, R. I.; Hermanson, G. T.; Mallia, A. K.; Gartner, F. H.; Provenzano, M. D.; Fujimoto, E. K.; Goeke, N. M.; Olson, B. J.; Klenk, D. C. (1985) Measurement of protein using bicinchoninic acid. Anal. Biochem. 150: 76-85). Pieces of c gelatin cryogel were removed mechanically from the carrier and freeze dried; different weights of the gel were suspended in 0.5 ml water with thorough vortex shaking. To different amounts of the gelatin gel suspension was added 1 ml of BCA solution and the mixture was incubated at 37° C. for 30 min. The absorbance was measured at 562 nm after centrifuging the samples. Appropriate controls were taken using native acrylamide gels. For comparison, dried gelatin gel pieces were also directly treated with BCA solution and the absorbance read for the supernatant. The standard curve was made by quantitative additions of gelatin to the native acrylamide cryogel and absorbance measured under the same conditions.

### EXAMPLE 21

### Sterilisation of the Gelatine-Cryogel Minicolumns for Cell Culture

The column packed with cryogel-containing minicolumns according Example 20 was washed with 30% ethanol (500 ml) under sterile conditions for 1 h. The 30% ethanol solution was then replaced by 70% ethanol (500 ml) and gel kept under shaking for 2 h under sterile conditions. The gel was then washed extensively with sterile water to remove any traces of ethanol. Gelatin-cryogel affinity column was then allowed to equilibrate in sterile PBS.

### EXAMPLE 22

### Preparation of Cu(II)-IDA Acrylamide Cryogel Adsorbent

The epoxy-containing supermacroporous monolithic cryogel of Example 21 was washed with 30 ml of 0.5 M Na₂CO₃ for 30 min. Then the monolith was equilibrated with IDA solution (0.5 M in 1M Na₂CO₃, pH 10.0; 30 ml). The gel was then incubated in IDA solution for 48 h at room temperature with continuous shaking. The IDA acrylamide cryogel was then washed thoroughly with di-stilled water until pH reached 8.0. Copper was loaded on to the IDA cryogel by adding 30 ml of 0.1 M CuSO₄ solution to the monolith. Coupling was allowed to proceed for 2 h. Finally the gel was washed thoroughly with water to remove any unbound copper. Then the gel was washed with imidazole buffer (15 mM in 20 mM Hepes and 0.2 M NaCl, pH 7.0) to remove loosely bound copper.

Growth of Adherent Human Cell Lines on Gelatin-Cryogel Matrix

### EXAMPLE 23

### Culturing Human Fibrosarcoma Cell Line HT1080

Human fibrosarcoma cell line HT 1080 was cultured in DMEM containing 10% fetal calf serum, 1.2 mg/ml sodium bicarbonate, 10 KIU/ml aprotinin and 0.2% (v/v) kanamycin.

The sterile gelatin-cryogel of Example 21 (100 ml bed volume) was washed with 500 ml of sterile phosphate buffered saline (PBS). The gel was then equilibrated with 300.0 ml of the culture medium. Human kidney cells HT1080 (5.0 ml, 1×10⁶ cells) suspended in the culture medium were applied to the gelatin-cryogel matrix and 100.0 ml flow through was collected before cells were allowed to run completely into the monolithic column bed. The column outlet was closed and cells were allowed to bind efficiently to the matrix by incubating the column at 37° C. in 5% carbon dioxide environment. A filtering (0.2 μm) inlet was provided in the column for the exchange of carbon dioxide from the COD incubator into the cell culture device. After a period of 6 hours, the cell culture device (gelatin-cryogel column with attached cells) was connected to 500 ml media reservoir containing DMEM media supplemented with 1.2 mg/ml sodium bicarbonate, 10 KIU/ml aprotinin and 0.2% (v/v) kanamycin and 2 or 10% fetal calf serum. The media from the reservoir was circulated through the column at a low flow rate of 4 ml/min. Initial flowthrough (100.0 ml) from the gelatin-cryogel cell culture device was collected and analysed for the presence of unattached cells. For the continuous production of biomolecule (urokinase), the cell culture bioreactor device was continuously run for 18 days by circulating the medium over the reactor. For the integrated capture of the biomolecule, a sterile Cu(II)-IDA polyacrylamide cryogel column (bed volume 5.0 ml) for urokinase capture was connected as the production level of the biomolecule reached to the maximum. The capture column(s) was placed in between the cell culture device and the media reservoir. Whole integrated cell culture bioreactor set-up was continuously run for 4 weeks. Two ml samples were subsequently withdrawn from the cell culture device at regular intervals (24 h) to monitor the growth of cells and secreted protein.

### EXAMPLE 24

### Cell Growth and Product Secretion by HT1080 Cell Line

The amount of gelatin immobilized on the cryogel matrix of cryogel-containing minicolumn prepared according to Example 21 was 2.1 mg gelatin/ml cryogel. After allowing the cells to grow on the cell culture device for about four weeks, the cryogel matrix with the cells was removed mechanically from the carrier and disengaged into pieces of 1-2 mm in size. The cells were removed from the cryogel by treating with 0.2% trypsin-EDTA solution. The cell number and viability was checked using trypan blue dye exclusion method. A considerable number of cells could be seen adhering to the gelatin-cryogel even after the trypsin-EDTA treatment, making it difficult to have an accurate cell count on the number of cells growing on the cryogel monolith. However, the cells recovered and counted gave the cell number of 1.5×10⁸ cells. The cells eluted from the cryogel were then inoculated in a T-flask. It was observed that cell attachment to the T-flask surface took somewhat longer time period (20-24 h) as compared to that for routine cell cultures (3-5 h). However, the adherence property of the cells was well retained. A confluent monolayer was formed in 7 days. No morphological changes were observed in the cells eluted from the gelatin-cryogel monolith and re-cultured in the normal cell culture flask. Normal production of protein product from cells detached from the matrix was observed. The cells grow on the gelatin-cryogel scaffold as a tissue sheet and were analysed using scanning electron microscopic studies (FIG. 2).

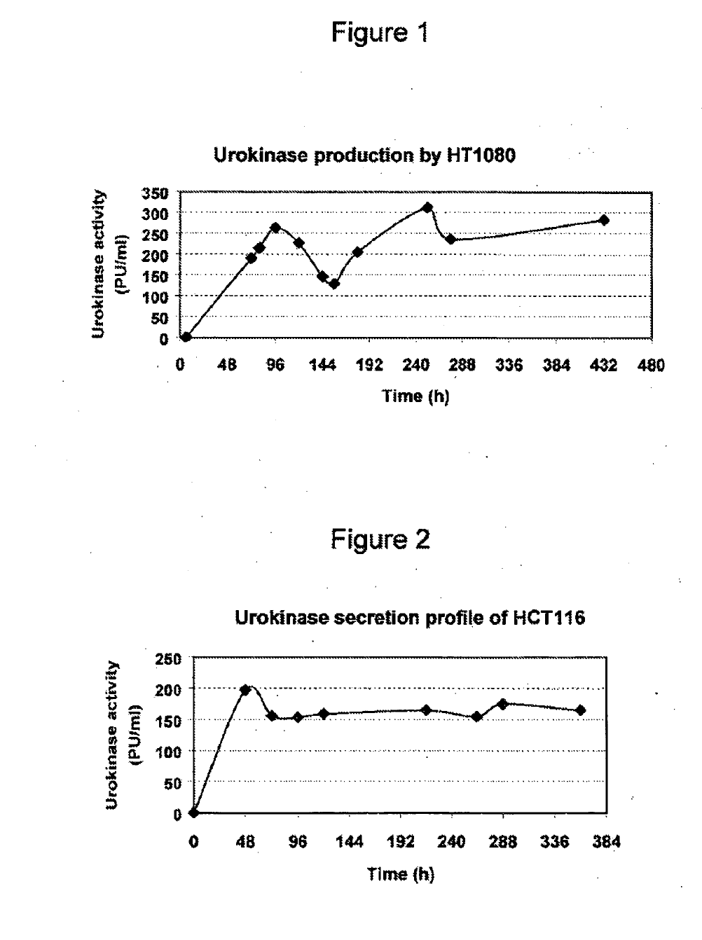

FIG. 1 shows the continuous production of urokinase by cells grown on the gelatin-cryogel cell culture device. The cells were grown continuously on 100 ml column packet with cryogel-containing minicolumns for 18 days. The media was circulated through the cells cultivated on matrix at a flow rate of 4 ml/min and samples withdrawn at regular intervals for determination of the urokinase activity and any detached cells from the matrix scaffold. The production level increased sharply within the first 96 h of incubation and overall, production level increased to about 300 PU/ml of the culture supernatant after continuously running the cell culture bioreactor for 18 days. In between decreases were observed in the urokinase levels. This probably may be because of the increased level of plasminogen activator inhibitor secretion by the cell line (Wun T C; Palmier M O; Siegel N R; Smith C E (1989) Affinity purification of active plasminogen activator inhibitor-1 (PAI-1) using immobilized anhydrourokinase. Demonstration of the binding, stabilization, and activation of PAI-1 by vitronectin. (J. Biol. Chem. 264: 7862-8.). The inhibitor will form the complex with the urokinase and that makes it difficult to estimate the actual level of the enzyme.

### EXAMPLE 25

### Culturing Human Colon Carcinoma Cell Line HCT116

Human colon carcinoma cell line HCT116 was cultured in McCoy's medium containing 10% fetal calf serum and 0.2% (v/v) kanamycin.

The sterile gelatin-cryogel of Example 21 (4-5 ml bed volume) was placed in a plastic container such that all liquid had to pass through the gel and washed with 30.0 ml of sterile phosphate buffered saline (PBS). The gel was then equilibrated with 15.0 ml of the culture medium. Human colon carcinoma cell line HCT116 (5.0 ml, 1×10⁶ cells) suspended in the culture medium was seeded to the gelatin-cryogel matrix and 5.0 ml flow through was collected. The column outlet was closed and cells were allowed to bind to the matrix by incubating the column at 37° C. in 5% carbon dioxide environment. A filtered (0.2 μm) inlet was provided in the column for the exchange of carbon dioxide from the COD incubator into the cell culture device. After a period of 6 hours, the gelatin-cryogel column with attached cells was connected to 500 ml media reservoir containing McCoy's medium supplemented with 10% fetal calf serum and 0.2% (v/v) kanamycin. The media from the reservoir was circulated through the column at a flow rate of 0.2 ml/min. Initial flow through (5.0 ml) from the gelatin cryogel column was collected and analysed for the presence of unattached cells. For the continuous production of biomolecule (urokinase), the cell culture bioreactor device was continuously run for 15 days. Two ml samples were subsequently withdrawn from the cell culture device at regular interval (24 h) to monitor the growth of cells and excreted protein.

As a control non-adherent cell line BB5.1 Mouse anti-mouse C5, secreting monoclonal antibodies was applied to the gelatin-cryogel disc (2 mm thick and 10 mm diameter) in multiwell cell culture plate. The cells were allowed to grow in DMEM Glutamax-I medium with 10% FCS and containing 0.2% (v/v) kanamycin. The cells did not adhere to the gelatin-cryogel scaffold. The cells however, were grown like normal suspension culture similar as growth observed in normal tissue culture flask.

### EXAMPLE 26

### Cell Growth and Product Secretion by HCT116 Cell Line

No cells were observed coming out from the cell culture device of Example 25 after initial 6 h of incubation without medium circulation. Live cells were intermittently observed coming out from the cell culture device after 8 days of the growth, indicating full confluence on the cryogel cell support. After allowing the cells to grow on the cell culture device for about 15 days, the cryogel matrix with the cells were removed from the syringe and discs (1-2 mm) were cut. The cells were removed from the cryogel by treating with 0.2% trypsin-EDTA solution. The cell number and viability was checked using trypan blue dye exclusion method. Higher cell numbers in the order of 5×10⁸ cells/5 ml of the gel were observed as compared to HT1080 cells line. Again a considerable number of cells remained unreleased from the gel. The cells also grow on the gelatin-cryogel scaffold as a tissue sheet and were analysed using scanning electron microscopic studies.

Many human carcinoma cell lines excrete urokinase plasminogen activator to different levels. HCT116 is reported to produce higher levels of plasminogen activators as compared to other carcinoma cell lines (Boyd, D., Florent, G., Kim, P. and Brattain, M. (1988). Determination of the levels of urokinase and its receptor in human colon carcinoma cell lines. Cancer Res. 48: 3112-3116) So in this case also we monitored urokinase as an excreted protein to assess the continuous growth and protein excretion profile of the cell line.

FIG. 2, shows the continuous production of urokinase by cells grown on the gelatin-cryogel cell culture device. The cells were grown continuously on 100 ml column packed with cryogel-containing minicolumns for 18 days. The media was circulated through the cells cultivated on matrix at a flow rate of 4 ml/min and samples withdrawn at regular intervals for determination of the urokinase activity and any detached cells from the matrix scaffold. The production level increased sharply within the first 48 h of cell growth and thereafter a constant level of urokinase was observed. Again the secretion of plasminogen activator inhibitors and product feedback inhibition made it difficult to observe the actual level of urokinase in the spent broth.

### EXAMPLE 27

### Integrated Production and Separation of Excreted Protein

An integrated set-up based on cryogel cell culture bioreactor of Example 26 and affinity capture of the excreted protein with continuous supply of the nutrients is described here. Human kidney cell line HT1080 was grown on the cell culture bioreactor and the bioreactor was run continuously for 32 days with media circulation supplemented with 2% FCS. The complete adherence of the cells on the gel matrix was observed within 6 h of incubation without media circulation. No cells were observed coming out of the gel in first 10 days of cell growth on the cryogel matrix. Thereafter intermittently cells were seen in the flow throw from the cell bioreactor indicating confluence of the cells on the gel matrix. However, considerable number of live cells start appearing in the samples being collected from the setup after 15 days indicating that the column is fully saturated with cells around this point.

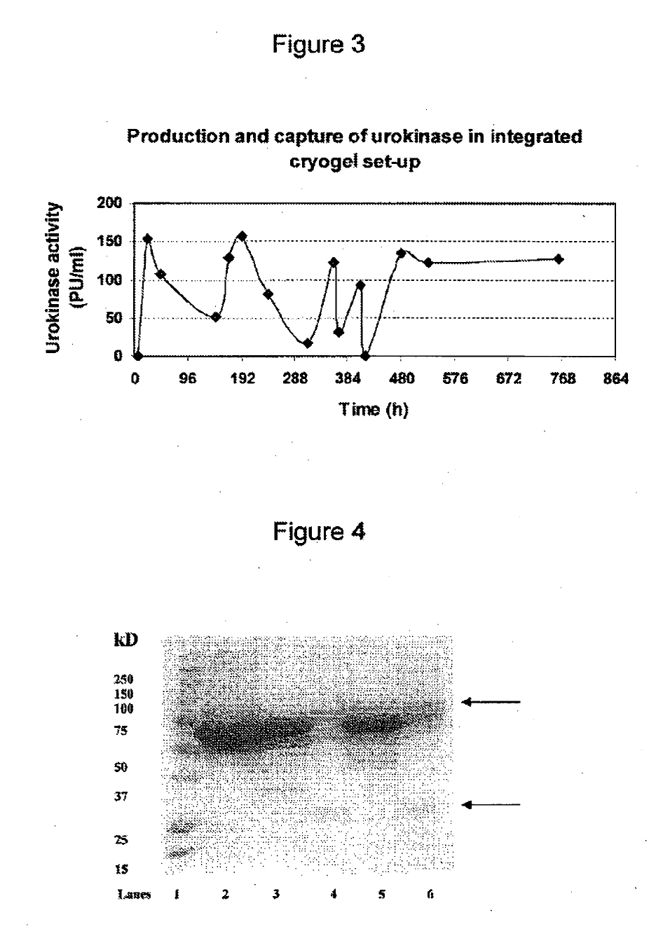

FIG. 3, presents the complete profile of the continuous urokinase secretion by the cell line with simultaneous recovery of the protein product using integrated affinity capture step. The cells were grown on the cryogel matrix for 32 days. The urokinase produced was captured on 50 ml Cu²⁺-AAm cryogel matrix. The arrows in the figure depict change of protein capture column. The thick block arrows at 360 and 408 hrs indicate integration of two 50 ml capture columns simultaneously in series. After 368 h of continuous run at a flow rate of 0.2 ml/min, the concentration of cell debris in the media reservoir and the urokinase production column increased significantly as observed by the change in media color as well as from microscopic examination of the samples from the production column. The media reservoir was thus replaced with a new one after 432 h. Samples were withdrawn from the production column and the capture columns for the determination of urokinase activity and any detached cells from the matrix scaffold (minicolumns). From the urokinase production profile and simultaneous capture of the enzyme, it is clear that the protein product is secreted continuously from the cell culture device. As the protein capture adsorbent removed the excreted protein from the circulatory media, the level of urokinase dropped significantly in the media broth. This level increased again as the capture column is removed from the set-up indicating constant release of the protein product and also decrease in the feedback inhibition. After changing the media on 18^{th} day, the cell culture bioreactor again produced the same level of urokinase as in the beginning. The overall level of the urokinase (about 150 PU/ml) was lower in this case as compared to HCT1080 bioreactor of Example 24, which gave about 300 PU/ml. This can be because of lower serum levels (2% FCS) in medium used in this case as compared to usual 10% FCS which secreted higher levels of the protein. FIG. 4, presents the SDS-PAGE of the captured protein product in the integrated set-up. In the figure there is shown:

Lane 1 Biorad prestained broad range protein marker

Lane 2 Load (10% serum containing media from HT1080 cell culture)

Lane 3 Break through fraction from Cu(II)-IDA Sepharose column

Lane 4 Peak fraction from Cu(II)-IDA Sepharose column (elution with 200 mM imidazole, pH 7.4)

Lane 5 Break through fraction from Cu(II)-IDA polyacrylamide cryogel column

Lane 6 Peak fraction from Cu(II)-IDA polyacrylamide cryogel column (elution with 200 mM imidazole, pH 7.4)

Arrows indicate low molecular weight and high molecular weights forms of urokinase

The protein recovered from the gel showed low molecular- and high molecular-weight forms of the urokinase. The major protein albumin in the medium remains unbound on the affinity capture filter and thus is re-circulated in the medium.

### REFERENCES

Mao D., Edwards J. R., Kuznetsov A. V. and Srivastava R. K. (2004) Three-dimensional numerical simulation of a circulation fluidized bed reactor for multi-pollutant control. Chemical Engineering Science 59, 4279-4289.

Masque N., Marce R. M. and Borrull F. (1998) New polymeric and other types of sorbents for solid-phase extraction of polar organic micropollutants from environmental water. Trends in Analytical Chemistry 17(8), 384-394.

Orlando U. S., Okuda T., Baes A. U., Nishijima W. and Okada M. (2003) Chemical Properties of anion-exchangers prepared from waste natural materials. Reactive and Functional Polymers 55, 311-318.

Poole C. F. (2003) New trends in solid-phase extraction. Trends in Analytical Chemistry 22(6), 362-373.

Rossi D. and Zhang N. (2000) Automating solid-phase extraction: current aspects and future prospects. J of Chromatography A 885, 97-113.

1. A composite sorbent material comprising
A) a housing of a dense, insoluble material and
B) a porous monolithic polymer gel housed within said housing,
said housing being provided with openings therein for fluid communication between said porous monolithic polymer gel and the surroundings, and
said porous monolithic polymer gel occupying the inner space of said housing and being kept on place within said housing by mechanical means.

2. A composite sorbent material according to claim 1, wherein said mechanical means comprises irregularities on the internal surface of the housing.

3. A composite sorbent material according to claim 1, wherein said housing is provided with a plurality of openings distributed over the surface thereof, said openings functioning as said mechanical means by parts of said porous monolithic polymer gel extending thereinto.

4. A composite sorbent material according to claim 1, wherein said housing substantially is in the shape of an open-ended cylinder and said mechanical means comprises a lattice of bars defining a plurality of open-ended duct-shaped spaces within said housing and extending in a direction from one of the open ends of the cylinder to the other.

5. A composite sorbent material according to any of claims 1 to 4, wherein said porous polymer gel is of macroporous structure.

6. A composite sorbent material according to any of claims 1 to 5, wherein said porous polymer gel is a cryogel.

7. A composite sorbent material according to claim 6, wherein said cryogel comprises a product obtained by polymerizing an aqueous solution of at least one water-soluble monomer selected from the group consisting of:
N-substituted and non-substituted (meth)acrylamides;
N-alkyl substituted N-vinylamides;
hydroxyalkyl(meth)acrylates;
vinylacetate;
alkylethers of vinyl alcohols;
ring-substituted styrene derivatives;
vinyl monomers;
(meth)acrylic acid and salts thereof;
silic acids; and
monomers capable of forming polymers via polycondensation;
under freezing at a temperature below the aqueous solvent crystallization point, at which solvent in the system is partially frozen with the dissolved substances concentrated in the non-frozen fraction of solvent to the formation of a cryogel.

8. A composite sorbent material according to claim 6, wherein said cryogel comprises a product prepared by cryogelation of at least one synthetic or natural polymer in the presence of a chaotropic agent and, if necessary or desired, a cross-linking agent.

9. A composite sorbent material according to claim 8, wherein said at least one synthetic polymer is poly(vinyl alcohol) in a cross-linked form.

10. A composite sorbent material according to claim 8, wherein said at least one natural polymer is at least one member selected from the group consisting of polysaccharides and proteins.

11. A composite sorbent material according to claim 10, wherein said at least one natural polymer is selected from the group consisting of agarose, agar, carrageenans, starches, cellulose and their respective derivatives and chitosan.

12. A composite sorbent material according to any of claims 1 to 11, wherein said porous polymer gel has been modified by introducing a member selected from the group consisting of, ligands, charged groups and hydrophobic groups thereinto.

13. A composite sorbent material according to claim 12, wherein the ligand is selected from the group consisting of peptides, metal chelates, sugar derivatives, boronate derivatives, enzyme substrates and their analogues, enzyme inhibitors and their analogues, protein inhibitors, antibodies and their fragments and thiol containing substances.

14. A composite sorbent material according to any of claims 7 to 11, wherein said cryogel has been modified by introducing a filler selected from the group consisting of metals, metal oxides, ion exchange particles, hydrophobic adsorbents and molecularly imprinted polymers in the form of particles.

15. A composite sorbent material according to claim 14, wherein the polymerization has taken place in the presence of a template molecule, thereby forming a molecularly imprinted polymer.

16. A process for the preparation of a composite sorbent material as defined in claim 7, which process comprises polymerizing an aqueous solution of at least one water-soluble polymer selected from the group consisting of:
N-substituted and non-substituted (meth)acrylamides;
N-alkyl substituted N-vinylamides;
hydroxyalkyl(meth)acrylates;
vinylacetate;
alkylethers of vinyl alcohols;
ring-substituted styrene derivatives;
vinyl monomers;
(meth)acrylic acid and salts thereof;
silic acids; and
monomers capable of forming polymers via polycondensation;
under freezing at a temperature below the aqueous solvent crystallization point, at which solvent in the system is partially frozen with the dissolved substances concentrated in the non-frozen fraction of solvent to the formation of a cryogel within a housing of a dense, insoluble material having openings therein and being provided with mechanical means keeping the monolithic cryogel thus prepared on place.

17. A process for the preparation of a composite sorbent material as defined in claim 8, wherein at least one substance selected from the group consisting of synthetic and natural polymers are subjected to cryogelation in the presence of a chaotropic agent and if necessary, a cross-linking agent within a housing of a dense, insoluble material having openings therein and being provided with mechanical means keeping the cryogel prepared on place.

18. A process according to claim 17, wherein an alkaline aqueous solution of agarose is filled into the housing and frozen at a temperature within a range below −10° C. to enable the formation of cryogel of agarose.

19. A process according to claim 17, wherein an acid aqueous reaction mixture of poly(vinyl alcohol) and glutaric dialdehyde is filled into the housing and is frozen at a temperature within a range below 0° C. to enable the formation of a cryogel of cross-linked poly(vinyl alcohol).

20. A process according to claim 17, wherein an aqueous solution of chitosan is filled into the housing and cooled to a temperature within a range below 0° C. until said solution is frozen whereafter the chitosan is cross-linked by means of a solution of glutaric dialdehyde in ethanol at a temperature within a range below 0° C. to enable the formation of a cryogel of cross-linked chitosan.

21. A process according to any of claims 16-20, which process comprises the additional step of adding an aqueous solution of a gel-forming compound, either alone or mixed with fillers or cells, to a composite sorbent material prepared according to any of said claims 16-20 and cooling the mixture obtained to a temperature within a range below 0° C. until said solution is frozen, whereafter the double frozen cryogel thus formed is thawed.

22. The use of a composite sorbent material according to any of claims 1 to 15 for the removal of pollutants from gases or liquids.

23. The use of a composite sorbent material according to any of claims 1 to 15 for the enrichment or separation of molecules that bind to structures in the porous polymer gel.

24. The use of a composite sorbent material according to any of claims 1 to 15 which is loaded with a biocatalyst for bioconversion of substances in a medium to be contacted with the composite sorbent material.

25. The use according to claim 24, wherein the biocatalyst is a member selected from the group consisting of enzymes and cells.

26. The use of a composite sorbent material according to any of claims 1 to 15 for the immobilization of cells.

27. The use of a composite sorbent material according to any of claims 1 to 15 in cell culture.

28. The use according to claim 27, wherein a suspension of cells is applied to a column filled with a composite sorbent material according to any of claims 1 to 16 the column is incubated for a period sufficient to bind the cells efficiently to the composite sorbent material whereafter a culture medium is circulated through the column.

29. A method of culturing mammalian cells by continuously bringing a liquid culture medium in contact with said mammalian cells in a bioreactor containing a support material to which said mammalian cells are attached, characterized in that a composite sorbent material as defined in any of claims 1 to 15 is used as said support material, said mammalian cells being accommodated in the pores of said composite sorbent material.

30. A method of culturing mammalian cells according to claim 29, wherein liquid culture medium from a media reservoir is continuously circulated through the bioreactor.

31. A method of culturing mammalian cells according to claim 30, wherein culture medium leaving the bioreactor is passed through an affinity absorber comprising a composite sorbent material according to any of claims 1 to 15 in order to capture target products released from the bioreactor before the culture medium being returned to the media reservoir.
